(19) United States
(12) Patent Application Publication
(10) Pub. No.: US 2008/0214957 A1
(43) Pub. Date: Sep. 4, 2008

(54) Bone Marrow Aspiration Trocar

(76) Inventors: Yvan Verra, Avignon (FR); Borhane Slama, Avignon (FR); Hacene Zerazhi, Barbentanc (FR); Ollivier Verra, Avignon (FR); Florence Verra, Avignon (FR); Raphael Verra, Vendene (FR)

Correspondence Address: WILLIAM J. SAPONE;COLEMAN SUDOL SAPONE P.C., 714 COLORADO AVENUE, BRIDGE PORT, CT, 06605, US

(21) Appl. No.: 10/560,146

(22) PCT Filed: Jun. 10, 2004
(86) PCT No.: PCT/FR04/01446

§ 371 Date: Oct. 22, 2007

(30) Foreign Application Priority Data
Jun. 12, 2003 (FR) 03/07064

(51) Int. Cl. A61B 5/00

(52) U.S. Cl. 600/578

(57) ABSTRACT

The invention relates to a bone marrow aspiration trocar comprising an element in the form of a hollow needle, the end of said element being provided with a prehension body (20) having a handle with lugs (4), and an insertion end (22). According to the invention, said insertion end (22) is closed and has lateral aspiration holes (21), whereas the other, prehension end (20) comprises a housing (L) provided with a bottom surface (28) which is penetrated by a perforating rod (24), and used as a support, either for a removable lid (26) or a capsule which is under a vacuum (27) and has a sealed diaphragm (27a), for handling said trocar.

### TECHNICAL FIELD

The present invention relates to a device (trocar) for taking bone marrow samples.

### BACKGROUND

In the medical field it is often necessary to take a sample of bone marrow from a patient, for quantitative and/or qualitative analysis purposes, or for other purposes: transplantations, etc. Said sample taking operations are generally performed on three human body bones: the front iliac bone, the rear iliac bone or the sternum.

In order to carry out the aforesaid sample taking operation, after the patient has been prepared, and particularly subjected to asepsis treatment, before the marrow can be reached, it is necessary to cross the patient's skin (both epidermis and derm), and then the periosteum and the outer part of bone called outer table. Great care has to be taken in order to prevent crossing the bone inner table, located over the marrow, as some other organs lie back to said bone.

Both the bone and the marrow have variable thickness, depending on the individual and on the exact collecting point. This sample taking operation requires then a long experience, great operator's skill and practice in using an instrument called a marrow sampling trocar.

There are widely known different kinds of trocars, both re-usable (metallic) or single-use, disposable ones (plastic with a metallic needle). FIG. 1 shows an embodiment of such a kind of instrument known in the art. In the aforesaid FIG. 1, a trocar 1 for bone piercing comprises a hollow body forming, at one end, a metallic hollow tube 2 shaped like a hollow needle, and at the opposite end a conic prehension body 20. This latter is provided with lugs 4 which define an upper surface 6.

Before the needle 2 is used to penetrate into the patient's body, an obstructing rod 3 must be inserted into the needle. This rod 3 has a funnel provided with a thrust surface 7 located at the center of the trocar body surface. The above rod prevents some materials (skin, bone fragments, etc.) from entering the needle, as these materials could otherwise obstruct the trocar 1 and subsequently prevent the marrow from being sucked up into the same needle by means of a manually operated sucking syringe 9 provided with a piston 9a. This sucking syringe 9, together with its manually operated piston, allows the operator to suck up the bone marrow 4 after the obstructing rod 3 has been removed, and thus replaced with the aforesaid sucking syringe.

The operator's actions consist of: inserting the obstructing rod 3 into the trocar 1; selecting a site for piercing the bone; arranging the trocar needle 2; pushing-for penetrating the skin 11 and then the periosteum 12 and the bone outer table 13, down to the marrow pockets 14, without piercing the back part 15 of the bone, called the inner table.

When the operator deems that the end of the trocar needle is positioned in a zone allowing the marrow to be sucked up, he stops pushing, then he removes the obstructing rod 3, places the syringe 9, and finally he attempts to suck up the marrow by hand, pulling the piston 9a.

Whatever is the operator's skill and experience, locating the sucking zone is a delicate and most of all depends on chance or luck to complete the operation. It is very frequent that pulling up the piston proves to be completely unsuccessful in sucking up the marrow.

In case of failure, the operator must repeat, often for several times, the whole series of operating steps, that is, the syringe must be removed, the obstructing rod must be placed into the trocar, the same must be pushed for penetrating the bone a little bit deeper, then the pushing action is stopped, the obstructing rod is removed, the syringe is re-positioned, there is a new try to suck up the marrow, and so on. These sequences of operating phases lead to possible detrimental consequences:

the infection risks are greatly increased due to the protraction of the sampling action, and to the subsequent and long exposure of the components to the air;

loss of precious time by the operator and his assistant, as some operations can take up to twenty minutes to complete;

increase of the patient's pain, that protracts for a long time;

sometimes inserting the obstructing rod into the trocar could be difficult.

The whole sampling operation, including the piercing steps, must be re-done from the start;

increased patient's pain, due to the new piercing operation.

In some extreme cases, by dint of pushing the needle ahead, there is a risk of perforating the bone inner table with, by consequence, a risk of perforating the underlying organs. Such an extreme case has been described in the past.

### OBJECTS OF THE INVENTION

The inventors have conceived a new trocar for taking samples of bone marrow which overcomes for the most part the above described drawbacks, thus improving the patient's comfort, while making the marrow sampling operation safer and decreasing the sampling duration time.

The present invention relates to a device for taking samples of liquid bone marrow, comprising an element in the form of a hollow needle, a prehension end of said needle being provided with a handle, and an opposite end thereof being an insertion end, wherein the insertion end is closed at its top and has lateral aspiration holes.

The trocar is closed and its insertion end could be pointed, chamfered or otherwise shaped, so as to fit to the sampling site on the patient, the trocart action thus becoming a divarication or spreading action, rather than a material removal action. This prevents the tissue or bone particles from separating from the surrounding tissues and from obstructing the aspiration channel.

Preferably, the hollow needle forms, at its prehension end, a hollow perforating rod, which communicates with a handle space, fit to penetrate a hermetic diaphragm of a vacuum chamber. This latter consists of a capsule acting both as a vacuum and aspiration source and as a collecting tank for the aspired liquid.

Other characteristic features and advantages of the present invention will appear evident from the following detailed description of an embodiment of the same invention.

### BRIEF DESCRIPTION OF THE FIGURES

Reference will be made to the enclosed drawings, wherein:

FIG. 1 shows a device according to the prior art;

FIG. 2 shows an embodiment of the device made according to the present invention.

### DETAILED DESCRIPTION OF THE INVENTION

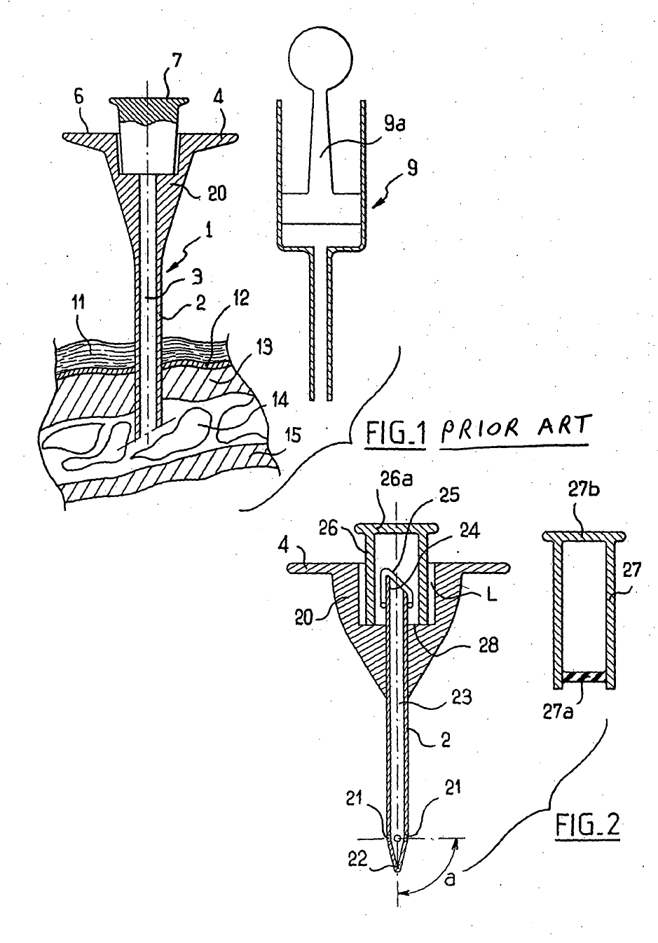

An embodiment of a trocar made according to the present invention, which is illustrated in FIG. 2, comprises a hollow needle, closed at its insertion end 22. This can be pointed or chamfered, so as its action becomes a divarication or spreading action, rather than a material removal action, thus preventing the tissue or bone particles from separating from the surrounding tissues and from obstructing the needle. The aforesaid closed end most of all allows the obstructing rod 3 of FIG. 1 to be eliminated. Therefore, it is not present in the new trocar.

The sample taking instrument is greatly simplified, as well as the operator's actions, since this latter is no longer obliged to remove the obstructing rod (as well as the steps od eventually, to replace it and to remove it again, as already described above), thus reducing the aforesaid risks (recalling: infection risks, time loss, patient's pain and more added pain).

The trocar according to the present invention is provided with one or more lateral aspiration holes 21, located over the closed end 22. These holes do not come out directly on the pushing axis, but they are inclined with respect with the same axis by an angle at least equal to 90°. In this way they do not allow small tissue particles to enter the needle.

The diameter of each one of the aforesaid holes 21 has been defined so as to be preferably smaller than the inner diameter of the needle 23 of the new trocar. Therefore, even in a very improbable event where some small tissue particles enter one of the holes, it would be impossible for them to obstruct the inner channel of the trocar needle 23, said channel having a diameter bigger than the aspiration holes 21.

However, the trocar made according to the present invention is provided, at its opposite end, with a hollow perforating rod 24, covered by a cap 25 for asepsis purposes.

Around the hollow perforating rod 24, a prehension body or handle 20 is provided, having horizontal lugs 4, the handle 20 comprising a housing L, provided with a bottom surface forming a support surface 28 for a removable cover 26, which can be arranged for closing the housing L, in order to give the operator an upper pushing plate 26a. The upper pushing plate 26a allows the operator to strongly prop his hand on the trocar and then to perforate the bone outer table.

The cover 26 is removed for positioning in its place, inside the housing L, a capsule 27, which leans toward the surface 28. A diaphragm 27a has been previously perforated during this positioning operation by the rod 24. The capsule 27 forms a chamber under partial vacuum, communicating with the trocar up to the aspiration holes 21. It has an upper pushing plate 27b similar to the one present in the cover 26.

Thanks to the present invention, the operator's action becomes considerably different, being optimized and rendered safe, as the following description will prove.

The operator inserts the trocar in the outer table of a bone 13 until the lateral aspiration holes 21 trespass its outer surface. Then it removes the cover 26, and substitutes it with the transparent capsule 27 under vacuum in the housing L. Subsequently, he continues with the penetration action.

When the lateral aspiration holes 21 come into a first contact with the marrow, then it will be automatically and instantly aspired by means of vacuum which is present in the trocar and in the capsule 27, where the marrow will immediately appear and it will be immediately seen by the operator. He then stops his push, and his unique subsequent task will be that of controlling the amount of marrow which is taken into the capsule 27.

Simply by changing a full capsule with one or more empty ones, the operator can very quickly perform some other sample takings, keeping an optimal aseptic condition, and with an evident improvement in the patient's comfort, since the operation time is very short. Moreover, inside the capsule, the sample is insulated by the surrounding atmosphere, thus ensuring a good sample quality.

Finally, it has to be noted that filling several capsules 27 in a very short time allows continuous instant analysis to be carried out with an optimum asepsis condition.

There are several advantages coming from the present invention. Among these:

the marrow sampling is optimized;

the risks of infection are reduced by the instant sample taking, without exposing the samples to the open air;

the possibility of re-inserting the obstruction rod into the marrow heart, after it has been exposed to the open air while carrying out unsuccessful sample taking operations, is substantially eliminated;

a saving of precious operator's time, and for his assistant and for other patients waiting for their turn;

a reduction in the pain duration for the patient, thanks to the operations quickness, and the suppression of every additional pain due to the subsequent instrument manipulation during the unsuccessful insertions and re-insertions of the obstructing rod 3;

the option to use several different capsule models can be provided, in order to immediately adapt the marrow samples to the different analysis techniques (this is not possible at all with the piston syringes provided with the trocars of the prior art). In this way, each capsule can have a particular shape, or a particular sign or colour indicating its final destination.

It has to be understood that needles of different length can be provided depending on the different sites where the marrow sampling has to take place; a short taking needle will be provided when a flat bone must be sampled, as the sternum; a longer needle will be provided when an intervention on a bone like the iliac bone has to be carried out.

We claim:

1. A device for taking liquid bone marrow samples, comprising an element shaped as a hollow needle, having at one end a prehension body including a handle (4) and an insertion end (22) at the other end, said device being characterized in that the insertion end (22) is closed at its top and has lateral aspiration holes (21), and in that said hollow needle forms, at its prehension end, a hollow perforating rod (24), protruding into a housing (L) of the prehension body (20), fit to pierce a watertight diaphragm (27) of a vacuum chamber (26).

2. The device according to claim 1, characterized in that the diameter of said lateral aspiration holes (21) is smaller than the inner diameter of said needle.

3. The device according to claim 1 or claim 2, wherein an axis of each aspiration hole (21) forms, with respect to a penetration axis of said needle (23), an angle of at least 90°.

4. The device according to claim 1, further comprising a removable cap (25) of the needle perforating rod, adapted to said housing (L) of the prehension body (20), in order to provide the operator with a pushing surface across said housing.

5. The device according to claim 1, wherein said vacuum chamber has an outer shape fit to be inserted in the housing of said prehension body (20), in order to provide the operator with a pushing surface across said housing (L).

6. The device according to claim 1, further comprising a series of vacuum chambers shaped as capsules (26), said capsules being different one each other, to designate each final destination.
